(19) United States
(12) Patent Application Publication
(10) Pub. No.: US 2024/0147962 A1
(43) Pub. Date: May 9, 2024

(54) WEARABLE DEVICE FOR MONITORING AND CONTROLLING BEHAVIOR OF PET

(71) Applicant: LaShaun Gray CHAPMAN, Burke, VA (US)

(72) Inventor: LaShaun Gray CHAPMAN, Burke, VA (US)

(21) Appl. No.: 18/054,140

(22) Filed: Nov. 9, 2022

(51) Int. Cl. A01K 15/02; A01K 27/00; A61B 5/00; A61B 7/00; G08B 3/10; H04R 1/02

(52) U.S. Cl. CPC A01K 15/022; A01K 27/006; A61B 5/6802; A61B 7/00; G08B 3/10; H04R 1/02; A61B 2503/40

(57) ABSTRACT

Various embodiments provide a wearable device for monitoring and controlling behavior of a pet. The wearable device includes a wearable element configured to be worn by the pet. The wearable device also includes at least one sensor arranged on the wearable element. The at least one sensor includes at least one biometric sensor operable to sense biometric-parameters based on biometric signals of the pet. The wearable device further includes a processor arranged on the wearable element and operatively coupled to the at least one sensor. The processor is configured to receive the biometric-parameters from the at least one biometric sensor and communicate the biometric-parameters to a user device for enabling a user to monitor and control behavior of the pet.

### TECHNICAL FIELD

The present disclosure relates, generally, to pet management, more particularly to, a wearable device and a system for monitoring and controlling behavior of a pet.

### BACKGROUND

A well-behaved pet, such as a dog, is the dream of every pet owner. The behavior of the pet typically depends on the relationship shared by the pet owner with the pet. Mostly, the relationship of the pet owner with the pet depends on how well the pet owner trains the pet and/or treats the pet. Training a pet is typically a very long, and tedious task. For example, it takes a month's effort for a dog owner to learn to properly walk with their dog without pulling a leash tied to the dog. Further, as we know that a dog's ability to smell is far superior to humans, and for this reason, when pet owners have a reactive or an aggressive dog, they worry about taking their dogs out of the house because of the fear of encountering another dog. Generally, there are various ways of training techniques, such as dog training devices, dog training courses, and so forth, available in the market by which a dog owner can train his/her dog.

However, the existing training techniques are not good enough for training or controlling the behavior of the dog. For example, there are several vibrations or shocking collars available in the market for training and/or correcting dogs' behavior. The major challenge with such vibration or shocking collars is that the dog owner typically operates them. Specifically, an uneducated or unaware dog owner lacks significantly in terms of controlling the timing of activating and deactivating such collars. For example, if the dog is not displaying any unruly or undisciplined behavior (like, aggressively pulling the dog owner or barking at someone) and the dog owner activates the collar. In such situations, the dog gets confused and disturbed, which in turn can act as a negative behavioral reinforcement for the dog. Further, the issue with the dog owners is that they do not pay enough attention to monitoring or are not aware enough to understand dog communications, which is expressed by the dog using its tail, ears, mouth, and eyebrows. In such instances, training, monitoring, or controlling the behavior of the dog becomes even more challenging. The existing training techniques lack the consideration of the dog's communication and/or feelings while being applied.

In light of the foregoing discussion, there exists a need to overcome the aforementioned drawbacks associated with conventional training techniques for monitoring and controlling the behavior of a pet.

### SUMMARY

Various embodiments of the present disclosure provide a wearable device for monitoring and controlling the behavior of a pet. The wearable device enables both the pet and the pet owner to be trained for managing the pet when subjected to stress and control the behavior of the pet when subjected to high stress.

In an embodiment, a wearable device for monitoring and controlling the behavior of a pet is disclosed. The wearable device includes a wearable element configured to be worn by the pet. The wearable device also includes at least one sensor arranged on the wearable element. The at least one sensor includes at least one biometric sensor operable to sense biometric-parameters based on biometric signals of the pet. The wearable device further includes a processor arranged on the wearable element and operatively coupled to the at least one sensor. The processor is configured to receive the biometric-parameters from the at least one biometric sensor and communicate the biometric-parameters to a user device for enabling a user to monitor and control the behavior of the pet.

In another embodiment, a system for monitoring and controlling behavior of a pet is disclosed. The system includes a wearable device having a wearable element configured to be worn by the pet. The wearable device also includes at least one sensor arranged on the wearable element, the at least one sensor includes at least one biometric sensor operable to sense biometric-parameters based on biometric signals of the pet. The wearable device further includes a processor arranged on the wearable element and operatively coupled to the at least one sensor, the processor is configured to receive the biometric-parameters from the at least one biometric sensor. The system also includes a user device communicably coupled to the wearable device for receiving the biometric-parameters, the user device notifies the biometric-parameters to a user for enabling the user to monitor and control behavior of the pet.

Other aspects and example embodiments are provided in the drawings and the detailed description that follows.

### BRIEF DESCRIPTION OF THE FIGURES

For a more complete understanding of example embodiments of the present technology, reference is now made to the following descriptions taken in connection with the accompanying drawings in which:

FIG. 1 is a block diagram of a wearable device for monitoring and controlling behavior of a pet, in accordance with an embodiment of the present disclosure;

FIG. 2 is a block diagram of a wearable device for monitoring and controlling behavior of a pet, in accordance with another embodiment of the present disclosure;

FIG. 3 is a block diagram of a system for monitoring and controlling behavior of a pet, in accordance with an embodiment of the present disclosure;

FIGS. 4A and 4B are schematic front and rear views of the wearable device of FIG. 2, in accordance with an embodiment of the present disclosure;

FIG. 5 is a schematic illustration of the wearable device of FIG. 4A worn by a pet, in accordance with an embodiment of the present disclosure; and

FIG. 6 is a block diagram of a system for monitoring and controlling behavior of a pet, in accordance with another embodiment of the present disclosure.

The drawings referred to in this description are not to be understood as being drawn to scale except if specifically noted, and such drawings are only exemplary in nature.

### DETAILED DESCRIPTION

In the following description, for purposes of explanation, numerous specific details are set forth in order to provide a thorough understanding of the present disclosure. It will be apparent, however, to one skilled in the art that the present disclosure can be practiced without these specific details.

Reference in this specification to “one embodiment” or “an embodiment” means that a particular feature, structure, or characteristic described in connection with the embodiment is included in at least one embodiment of the present disclosure. The appearance of the phrase “in an embodiment” in various places in the specification is not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

Moreover, although the following description contains many specifics for the purposes of illustration, anyone skilled in the art will appreciate that many variations and/or alterations to said details are within the scope of the present disclosure. Similarly, although many of the features of the present disclosure are described in terms of each other, or in conjunction with each other, one skilled in the art will appreciate that many of these features can be provided independently of other features. Accordingly, this description of the present disclosure is set forth without any loss of generality to, and without imposing limitations upon, the present disclosure.

Referring now to the drawings, FIG. 1 is a block diagram of a wearable device 100 for monitoring and controlling behavior of a pet, in accordance with an embodiment of the present disclosure. As shown, the wearable device 100 includes a wearable element 102 configured to be worn by the pet, at least one sensor 104 arranged on the wearable element 102 and a processor 106 arranged on the wearable element 102 and operatively coupled to the at least one sensor 104.

In an embodiment, the wearable element 102 includes one of a vest, a harness, or a collar. Typically, the wearable element 102 includes any wearable configuration, which can be comfortably worn around a neck or a shoulder or a chest of the pet. Further, the wearable element 102 may be made of a suitable material like fabric, plastic, rubber, or any combination thereof. The wearable element 102 includes the wearable configuration that provides a sufficient surface area that enables in the mounting of the at least one sensor 104 and the processor 106 thereon. In an example, the at least one sensor 104 and the processor 106 may be detachably or integrally coupled to the wearable element 102. According to an embodiment, the at least one sensor 104 and the processor 106 may be combined to form an electronic unit. For example, the at least one sensor 104 and the processor 106 may be arranged on a printed circuit board to form the electronic unit. The electronic unit may further include other electronic components, such as transistors, resistors, capacitors, and integrated circuits required for desired operation or function of the at least one sensor 104 and the processor 106.

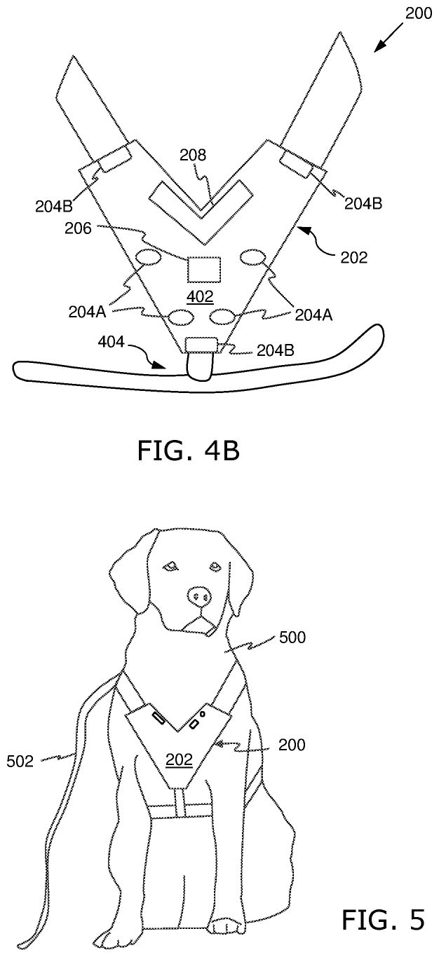

It will be evident to a person skilled in the art that the at least one sensor 104 may be arranged on the wearable element 102 in a manner such that the at least one sensor 104 is capable of reading or sensing the biometric signals of the pet when the wearable device 100 is worn by the pet. Typically, the wearable element 102 is configured to have a shape that conforms to a shape of the pet's body, and the at least one sensor 104 is arranged on the wearable element 102 in a manner such that the at least one sensor 104 can measure the biometric-parameters of the pet. In an example, the at least one sensor 104 may be arranged or placed on the lateral sides of the dog's chest or the rib cage to measure the biometric-parameters of the pet. An exemplary embodiment of the wearable element 102 is better explained in conjunction with FIGS. 4A and 4B, herein later.

The at least one sensor 104 includes at least one biometric sensor operable to sense biometric-parameters based on biometric signals of the pet. Typically, the at least one sensor 104 is configured to determine biometric-parameters, i.e., measurable values corresponding to the biometric signals, such as respiration rate or heart rate. According to an embodiment, the at least one biometric sensor may be operable, based on the principles of electrocardiogram (ECG), photoplethysmogram (PPG), or any combination thereof, to measure the biometric-parameters, i.e., the respiration rate or the heart rate.

According to an embodiment, the biometric-parameters, i.e., the measured respiration rate and/or the heart rate are used to determine a stress level of the pet. For example, an increased respiration rate and/or heart rate to a pre-determined high value may be indicative of a high stress level, similarly, a moderate respiration rate and/or heart rate of around a standard value may be indicative of a moderate stress level, and lower respiration rate and/or heart rate to a pre-determined low value may be indicative of a low or no stress level. It will be evident that the pet's behavior may alter based on the stress level subjected to the pet. For example, with an increased stress level, the pet may tend to behave unruly, or undisciplined, and such undesirable behaviors may be shown by the pet by way of aggressively barking and/or pulling the pet owner. Such situations may occur when a pet dog sees another dog or smells another dog and considers the other dog as an enemy dog or as a territorial challenge.

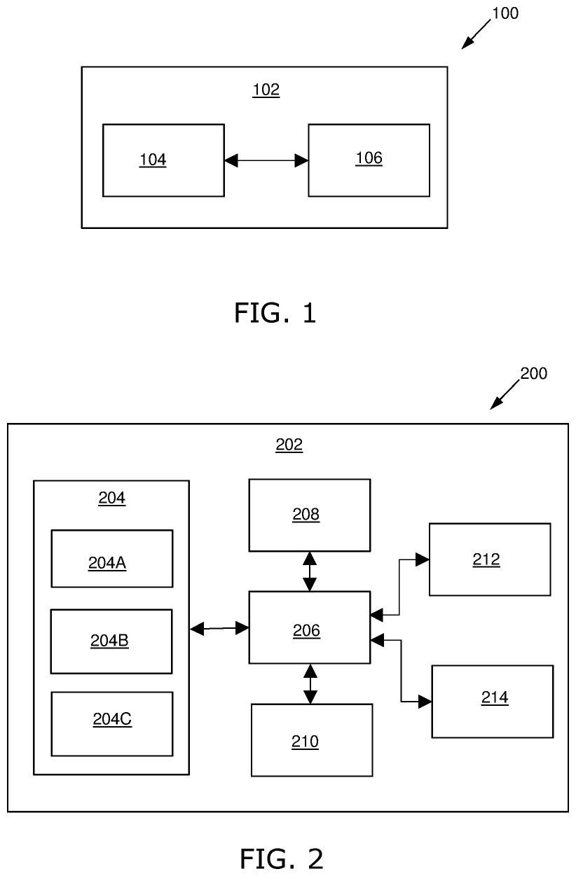

The processor 106 is configured to receive the biometric-parameters from the at least one biometric sensor and communicate the biometric-parameters to a user device (not shown in FIG. 1, best explained in conjunction with FIG. 5) for enabling a user to monitor and control behavior of the pet. Here, the term “user” may be referred to a pet owner specifically, and, to a person taking care of the pet. According to an embodiment, the processor 106 may be a microprocessor or a microcontroller. Further, the wearable device 100 may further include a communication interface (not shown in FIG. 1, best explained in conjunction with FIG. 2) for enabling communication coupling between the wearable device 100 and the user device. In an example, the communication interface may include electronic components, such as a transceiver (radio transmitter and receiver), a device that can both transmit and receive radio communications. Additionally, the communication interface may be designed to a specific standard, such as short message service (SMS), Bluetooth transmission, Wi-Fi transmission, which enables wireless communication between the wearable device 100 and the user device.

According to an embodiment, the biometric-parameters may be communicated to the user device based on a predetermined threshold value. For example, when the values of the biometric-parameters exceed a predetermined threshold value, which may be indicative of a high or moderate stress level, then only the wearable device 100 notifies the user device about such biometric-parameters. Alternatively, the biometric-parameters may be communicated to the user device based on real time, i.e., wherever the biometric-parameters value of the pet sensed by the wearable device 100 in real time is communicated to the user device.

As mentioned herein above, the biometric-parameters are communicated to the user device for enabling the user to monitor and control behavior of the pet. Essentially, the aspect of monitoring the behavior of the pet includes getting notifications regarding the biometric-parameters or the stress level of the pet over the user device. This allows the user to take appropriate actions, which can handle the stress level of the pet, thereby avoiding any display of unruly or undisciplined behavior by the pet. For example, the pet owner may establish physical contact with the pet by way of cuddling, hugging, caressing and the like to divert the pet's attention or to induce a safe and positive feeling in the pet. This allows the user to control the behavior of the pet, specifically in public places when the pet gets agitated due to the presence of any other animal. In other words, the notification regarding the stress level of the pet allows the pet owner to take appropriate actions for controlling the pet's behavior in public places or training the pet regarding how to behave in public places. Alternatively, the notifications regarding the stress level help the pet owner to better manage the pet's behavior and establish a trusting bond with the pet. Additionally, the notifications such as a continuous stress level without any external reason may indicate a poor health condition of the pet, for which the pet owner can take appropriate therapeutic action.

Referring now to FIG. 2, illustrated is a block diagram of a wearable device 200 for monitoring and controlling behavior of a pet, in accordance with another embodiment of the present disclosure. The wearable device 200 is substantially similar to the wearable device 100 of FIG. 1, for example, the wearable device 200 also includes a wearable element 202, such as the wearable element 102, at least one sensor 204, such as the at least one sensor 104, and a processor 206, such as processor 106. However, the at least one sensor 204 of the wearable device 200 includes at least one force sensor 204B and at least one sound sensor 204C in addition to at least biometric sensor 204A, explained herein above in conjunction with wearable device 100 of FIG. 1. The wearable device 200 also includes at least one vibrating element 208, a speaker 210, and a light source 212. The wearable device 200 further includes a communication interface 214, explained herein above in conjunction with wearable device 100 of FIG. 1.

The at least one force sensor 204B is arranged on the wearable element 202 and operatively coupled to the processor 206. According to an embodiment, the at least one force sensor 204B may be arranged on the wearable element 202 in a manner such that when the wearable element 202 is subjected to any pull force or tensile force, the at least one force sensor 204B responds to the applied force to converts the applied force into a measurable quantity. It would be evident to a person skilled in the art that the least one force sensor 204B, alternatively which can be termed as a pressure sensor, would be operable to convert the pull force or tensile force in a measurable quantity of pressure. In other words, when the wearable element 202 is stretched due to the application of force thereon, the at least one force sensor 204B is operable to measure force signals (or pressure signal or value) corresponding to a change in shape of the wearable element 202 due to the stretching. Therefore, the at least one force sensor 204B is operable to sense force or pressure signals when the pet applies a pull force on the wearable element 202 coupled to a leash (shown in FIG. 5) to be held by the user. In an example, the at least one force sensor 204B may be arranged on a portion, of the wearable element 202, adjacent to which the leash is coupled. This would allow the at least one force sensor 204B to suitably sense the force or pressure signals generated due to the application of the pull force by the pet on the wearable element 202 when the user is holding the leash.

According to an embodiment, the at least one force sensor 204B may be force-sensing resistors having a conductive polymer film that changes its resistance in a predictable manner when force is applied on its surface. Alternatively, the at least one force sensor 204B may be capacitive load cells or strain gauge load cells or pressure transducer, or piezoelectric sensor. As explained herein above in conjunction with FIG. 1, the at least one sensor 104 (i.e., the at least biometric sensor) and the processor 106 may be arranged on the printed circuit board to form the electronic unit. Similarly, the at least one force sensor 204B may be electrically coupled to the printed circuit board to form a part of the electronic unit. Further, the electronic unit may be arranged or coupled to the wearable element 202. Alternatively, the at least one force sensor 204B may be arranged separately or individually on the wearable element 202, and operatively coupled to the processor 206.

In an embodiment, the at least one vibrating element 208 is arranged on the wearable element 202 and operatively coupled to the processor 206. As explained herein above, similar to the at least one force sensor 204B, the at least one vibrating element 208 may be electrically coupled to the printed circuit board to form a part of the electronic unit. And the electronic unit may be coupled to the wearable element 202. Alternatively, the at least one vibrating element 208 may be arranged separately or individually on the wearable element 202, and operatively coupled to the processor 206.

The processor 206 is configured to receive the force or pressure signals from the at least one force sensor 204B to automatically activate the at least one vibrating element 208 for controlling the behavior of the pet. Typically, when the pet is subjected to any situation, which causes an increase in the excitement level of the pet and the pet tries to pull the pet owner holding the leash the wearable element 202 is subjected to the pull force. In such instance, the at least one force sensor 204B senses or measures the pull force subjected to the wearable element 202, and the sensed forced signals are communicated to the processor 206 to activate the at least one vibrating element 208. In an embodiment, the processor 206 is configured to activate the at least one vibrating element 208 only when the sensed forced signals exceed a threshold value of forced signals. This will avoid the activation of the at least one vibrating element 208, when the pet is gently or normally pulling the pet owner, unlike when pulling aggressively.

According to an embodiment, the at least one vibrating element 208 may include mechanical components and electrical components coupled to such mechanical components and operable to move the mechanical components for creating vibrations. Typically, the vibrations made by the at least one vibrating element 208 are deeply unpleasant for the pet and enable curtailing aggressive behavior of the pet. Additionally, the vibrations made by the at least one vibrating element 208 cause the pet to seek attention from the pet owner. Typically, the vibrations made by the at least one vibrating element 208 act as an indicative training signal to stop the agitated pulling action by the pet and to establish communication with the pet owner. In such instances, the pet owner can induce positive behavioral reinforcement in the pet by establishing training cues or words like “slow” that will begin to couple with the vibration in the pet's mind. Further, the aspect of automatically (without any manual intervention from the pet owner) activating the at least one vibrating element 208 by the processor 206 (based on the force or pressure signals sensed by the at least one vibrating element 208) allows for providing indicative training signals to the pet for stopping any agitative pulling action by the pet. Providing the pet with instant and consistent correction at the right time to establish a connection in the pet's mind between the vibration and the pressure or pulling cutting training down significantly. Additionally, the at least one vibrating element 208 is automatically deactivated by the processor 206 when the pet stops the agitative pulling action (i.e., when the sensed forced signals by the at least one force sensor 204B diminishes or is less than the threshold value of forced signals).

In an embodiment, the at least one sound sensor 204C is arranged on the wearable element 202 and operatively coupled to the processor 206. As explained herein above, like the at least one force sensor 204B the at least one sound sensor 204C may be electrically coupled or arranged on the printed circuit board to form a part of the electronic unit. Further, the electronic unit may be arranged or coupled to the wearable element 202. Alternatively, the at least one sound sensor 204C may be arranged separately or individually on the wearable element 202, and operatively coupled to the processor 206. According to an example, the at least one sound sensor 204C, may be one of the dynamic microphones, condenser microphones, ribbon microphones, and a carbon microphone. The at least one sound sensor 204C is operable to sense a barking sound of the pet. Typically, the at least one sound sensor 204C is operable to convert air pressure variations, caused by the barking sound, into electrical current or signals.

According to an embodiment, the processor 206 is configured to receive the barking sound from the at least one sound sensor 204C to automatically activate the at least one vibrating element 208 for controlling behavior of the pet. In an embodiment, the processor 206 is also configured to recognize a barking sound specific to a pet. This allows activating the at least one vibrating element 208 only when a concerned dog (mounted with the wearable device 200) barks and not when any other dog barks. The processor 206 is also configured to recognize a barking pattern or a barking sound intensity prior to automatically activating the at least one vibrating element 208. This allows activating the at least one vibrating element 208 only if the dog is barking in an agitated or aggressive manner, specifically when subjected to an increased stress level. The aspect of automatically activating the at least one vibrating element 208 based only on the barking sound also acts as an indicative training signal for the dog to stop agitative or aggressive barking behavior. Additionally, in such instances, the pet owner can establish communication with the pet by touching the pet, closing blinds/doors to calm the pet, or removing the pet from the agitating environment. Moreover, the at least one vibrating element 208 is automatically deactivated by the processor 206 when the pet stops the aggressive barking action (i.e., when the intensity of the sensed barking sound by the at least one sound sensor 204C diminishes or less than a threshold value of barking sound intensity that activates the at least one vibrating element 208). This gives the pet instant and consistent correction which can be coupled with a training cue like “quiet” that will become associated with and then take the place of the vibration in the pet's mind.

In an embodiment, the processor 206 is configured to correlate data of at least two of the at least one biometric sensor 204A, the at least one force sensor 204B, and at least one sound sensor 204C prior to automatically activating the at least one vibrating element 208. For example, the processor 206 may correlate the sensed or measured values of the at least biometric sensor 204A with the at least one force sensor 204B or the at least one sound sensor 204C prior to activating the at least one vibrating element 208. Alternatively, the processor 206 is configured to correlate all the sensors' data (measure values of the at least one biometric sensor 204A, the force sensor 204B, and the sound sensor 204C) prior to automatically activating the at least one vibrating element 208. This allows the processor 206 to activate the at least one vibrating element 208 only when the dog is subjected to increased stress level and displays an unruly or undisciplined behavior for which the dog owner needs to control the dog's behavior. In other words, the correlation of the sensors+ data enables avoiding false activation of the at least one vibrating element 208, which may act as negative behavioral reinforcement.

According to an embodiment, the wearable device 200 further includes the speaker 210 arranged on the wearable element 202 and operatively coupled to the processor 206. The speaker 210, like the sensors or the processor 206 or the at least one vibrating element 208, may be electrically coupled to the printed circuit board to form a part of the electronic unit. Otherwise, the speaker 210 may be mounted separately on the wearable element 202 and operatively coupled to the processor 206. The processor 206 is operable to activate the speaker 210 to generate a warning sound prior to activating the at least one vibrating element 208. The prior activation of the speaker 210 acts as a well-established operant conditioning training signal to warn and avoid the pet from displaying unruly or undisciplined behavior and induce positive behavioral reinforcement. Typically, when the at least one biometric sensor 204A, the force sensor 204B, or the sound sensor 204C senses a stress level but the stress level is not sufficient to activate the at least one vibrating element 208, in such instances the speaker 210 may be turned on to provide the warning sound for avoiding the pet from pulling or barking action.

In an embodiment, the wearable device 200 further includes the light source 212 arranged on the wearable element 202 and operatively coupled to the processor 206. The light source 212, similar to the speaker 210, may be electrically coupled to the printed circuit board to form a part of the electronic unit. Otherwise, the light source 212 may be mounted separately on the wearable element 202 and operatively coupled to the processor 206. In an example, the light source 212 may be at least one light emitting diode (LED). The processor is operable to activate the light source 212 upon activating the at least one vibrating element 208. Typically, when the at least one biometric sensor 204A, the force sensor 204B, or the sound sensor 204C indicates a stress level and the stress level is sufficient to activate the at least one vibrating element 208, in such instances the light source 212 is turned on by the processor 206. The turned-on light source 212 acts as an additional training signal to warn and avoid the pet from displaying unruly or undisciplined behavior, such as pulling or barking. It can also be a training cue for deaf dogs. Further, the light source 212 is turned on continuously till the time the pet is displaying the unruly behavior and once the pet stops the unruly behavior the light source 212 is turned off, and such action can induce positive behavioral reinforcement in the pet.

The wearable device 200 as shown in FIG. 2 also includes the communication interface 214, explained herein above in conjunction with wearable device 100 of FIG. 1. The communication interface 214 enables the wearable device 200 to communicate with the user device for notifying the stress level subjected to the pet. It will be evident to a person skilled in the art that the wearable device 200 includes a power source (not shown), such as a rechargeable or a non-reachable battery, for providing the required electrical power for the operation of various electronic components of the wearable device 200.

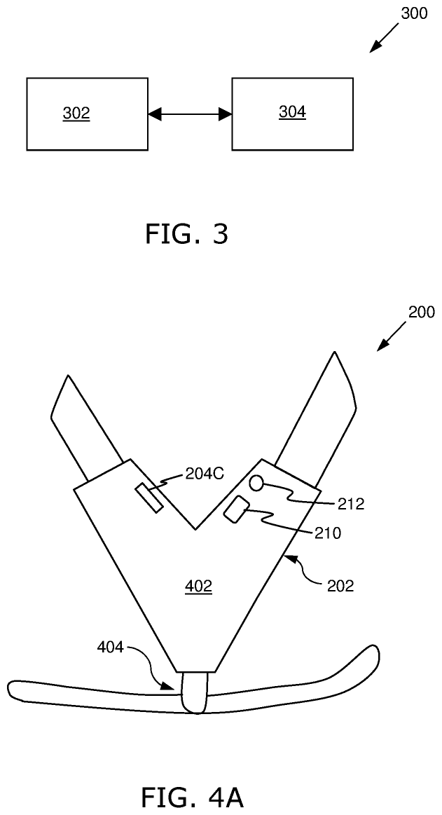

Referring now to FIG. 3, illustrated is a block diagram of a system 300 for monitoring and controlling behavior of a pet, in accordance with an embodiment of the present disclosure. As shown, the system 300 includes a wearable device 302 and a user device 304 communicably coupled to the wearable device 302. According to an embodiment, the wearable device 302 is one of the wearable devices 100 or the wearable device 200, and therefore the wearable device 302 is not explained in herein detail for brevity or to avoid repetition. In an embodiment, the user device 304 may be a portable wireless communication device, such as a mobile or a personal digital assistant, associated with the user.

The user device 304 is communicably coupled to the wearable device 302 for receiving the biometric signals. Further, the user device 304 notifies the biometric signals to a user for enabling the user to monitor and control behavior of the pet. According to an embodiment, the user device 304, which can be a mobile associated with the pet owner, may be notified by the wearable device 302 using Short Message Service (SMS). In an example, the wearable device 302 sends an SMS (or textual content) notification regarding the biometric signals. Typically, the SMS is displayed on a screen of the user device 304 regarding the stress level of the pet. For example, the stress level of the pet may be notified in terms of number (for example, higher or bigger the number on a scale of 10 or 100 higher is the stress level) or in terms of levels (such as low, moderate, or high). Additionally, the notification may be color-coded, for example, a red color notification for a high-stress level, a blue color notification for a moderate stress level, and a green color notification for a low-stress level. Accordingly, based on the notification (i.e., the stress level of the pet) the pet owner can communicate or interact with the pet. For example, if the notification indicates the high-stress level or moderate-stress level, the pet owner can immediately establish communication or interaction with the pet, such as physical contact with the dog by way of cuddling, hugging, caressing and the like, to divert the pet's attention or to induce safe and positive behavioral reinforcement in the pet.

In an embodiment, the user device 304 may be operable or configured to manually activate at least one vibrating element, such as the at least one vibrating element 208, of the user device 304 for controlling behavior of the pet. For example, based on the pet owner's choice, when the notification indicates that the stress level is moderate, then the pet owner may send an activation signal using the user device 304 to manually activate the at least one vibrating element 208. The notification, therefore, allows the user to activate the at least one vibrating element 208 only when the pet is subjected to a certain amount of stress. This enables avoiding any false or unwanted activation of the at least one vibrating element 208 by the pet owner, which can be disturbing and negative behavioral reinforcement for the pet. This also helps in training the pet owner to have a better sense of communication with the pet and to better understand the pet's behavior.

FIGS. 4A and 4B are schematic front and rear views, respectively, of the wearable device 200 of FIG. 2, in accordance with an embodiment of the present disclosure. As shown in FIG. 4A, the schematic front view of the wearable device 200 includes the wearable element 202 having a central portion 402 and a plurality of straps 404 coupled to the central portion 402 for mounting the central portion 402 on the pet (as shown in FIG. 5). As shown, the wearable element 202 is configured to be like a vest having the central portion 402 and the plurality of straps 404. Further, the wearable device 200 is shown to include the at least one sound sensor 204C, the speaker 210, and the light source 212 arranged on or coupled to the central portion 402 of the wearable element 202.

As shown in FIG. 4B, the schematic rear view of the wearable device 200 includes the wearable element 202 having the central portion 402 and the plurality of straps 404. Further, the wearable device 200 is shown to include the at least one biometric sensor 204A (i.e., four biometric sensors), the at least one force sensor 204B (i.e., three force sensors), the processor 206, and the at least one vibrating element 208 arranged on or coupled to the central portion 402 of the wearable element 202. As shown, the three force sensors (204A, 204B and 204C) and are arranged on the edges, of the central portion 402, to which the plurality of straps 404 is coupled. This allows the three force sensors 204B to suitably sense or measure the force or pressure signals when the pet applies the pull force on the wearable element 202 coupled to a leash (shown in FIG. 5) held by the user.

Referring now to FIG. 5, it is a schematic illustration of the wearable device 200 of FIG. 4A worn by a dog 500, in accordance with an embodiment of the present disclosure. As shown, the wearable device 200 is comfortably worn around a chest of the dog 500, so that the at least one sensor (not shown) is capable of suitably read or sense the biometric signals of the pet. Further, the wearable device 200, specifically the wearable element 202 thereof, is shown coupled to a leash 502.

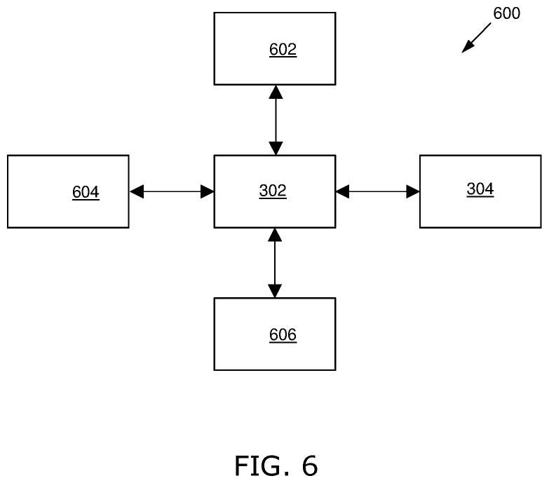

Referring now to FIG. 6, illustrated is a block diagram of a system 600 for monitoring and controlling behavior of a pet, in accordance with another embodiment of the present disclosure. The system 600 is similar to the system 300 (explained in conjunction FIG. 3). For example, the system 600 includes the wearable device 302 and the user device 304. However, the system 600 further includes a prohibitory sensor node 602, a portable vibrating fence 604 and a bell arrangement 606 communicably coupled to the wearable device 302 and/or the user device 304. The prohibitory sensor node 602 and the portable vibrating fence 604 are operable to prohibit the pet against accessing a prohibited area of the house. The bell arrangement 606 is operable to generate a tone when the pet sits or stands in front of the bell arrangement 606 for a pre-determined time period and notify the user device that the pet is in front of the bell arrangement 606.

According to an embodiment, the prohibitory sensor node 602 includes a proximity sensor, at least one of a light source or a sound source, and a power source. The prohibitory sensor node 602 may be implemented in a form of a portable wall mounted housing or a portable stand, enclosing the proximity sensor and the at least one of the light sources or the sound source therein. The prohibitory sensor node 602 is communicably coupled to the wearable device 302. The prohibitory sensor node 602 is kept in the or in the vicinity of a prohibited area, which can be any part of a house in which the access of the pet is not desired or not allowed.

In use, the prohibitory sensor node 602 is kept at such prohibited area of the house and when the pet approaches close to the prohibitory sensor node 602, the proximity sensor of the prohibitory sensor node 602 senses the approaching pet and activates either the light source or the sound source of the prohibitory sensor node 602 to generate a warning light signal or a warning sound signal to warn the pet against accessing the prohibited area. In case, the pet still tries to access the prohibited area, the prohibitory sensor node 602 sends an activation signal to the wearable device 302, which in turn activates the vibrating elements of the wearable device 302 worn by the pet. The warning light or sound signals from the prohibitory sensor node 602 or activation of the vibrating elements of the wearable device 302, trains (i.e., warns or prohibits) the pet against accessing the prohibited area of the house.

In an embodiment, the portable vibrating fence 604 includes a fence, a stand supporting the fence, a proximity sensor arranged in the stand, at least one of a light source or a sound source, a mechanical actuator coupled to the fence, and arranged in the stand and a power source. In use, similar to the prohibitory sensor node 602, the portable vibrating fence 604 may be kept in the prohibited area of the house. The portable vibrating fence 604 is communicably coupled to the wearable device 302. Therefore, if the pet approaches the portable vibrating fence 604, firstly either the light source or the sound source of the portable vibrating fence 604 is activated to generate a warning light signal or a warning sound signal, and secondly, the mechanical actuator is activated to vibrate the fence. This is to warn the pet against accessing the prohibited area of the house, however, if the pet still tries to access the prohibited area of the house, the proximity sensor of the portable vibrating fence 604 sends an activation signal to the wearable device 302, which in turn activates the vibrating elements of the wearable device 302 to prohibit the pet from accessing the prohibited area.

The prohibitory sensor node 602 and the portable vibrating fence 604 are helpful for managing dog's behavior in prohibited areas but also can address drinking from the toilets/pulling toilet paper, counter surfing, eating shoes, or when in new environments.

According to an embodiment, the bell arrangement 606 includes at least one bell (i.e., a speaker), a proximity sensor, and a power source. The bell arrangement 606 may be implemented in a form of a portable wall-mounted housing, which can be mounted on a door of the house. In use, the pet sits or stands in front of the bell arrangement 606, which will be sensed by the proximity sensor of the bell arrangement 606, and the bell arrangement 606 accordingly sends a notification to the user devise 304 (via the wearable device 302) that the pet is in front of the bell arrangement 606. The pet in front of the bell arrangement 606 may be an indication that the pet wants or needs to go outside the house. Typically, the bell arrangement 606 is operable to make a joyful or happy tone, when the pet sits in front of the bell arrangement 606 for the pre-determined time period, for example, 3 seconds or 5 seconds. The joyful or happy tone indicates to the pet that he/she will be taken out soon and at the same time the pet owner is notified about the pet in front of the bell arrangement 606. The sitting behavior is usually the first behavior trained and many puppies have accidents right next to the door. This will alleviate the number one reason for pets being rehoused—which is poor potty training.

Embodiments of the present disclosure substantially eliminate the top issues private dog trainers address in a consistent system guided by well-established operant conditioning protocols for monitoring and controlling the behavior of a pet. The bell, prohibitory sensor node, and the fence completely address potty training, counter surfing and a range of other issues that have not adequately been addressed previously. Typically, the wearable device and system help in notifying dogs' stress levels, which can be an indication of dogs' communication and/or feelings, to the dog owner and allowing the dog owner to perform desirable actions to manage the stress levels on time. This enables inducing positive behavioral reinforcement in the dog. The wearable device and system of the present disclosure accordingly help in training the pet owner regarding the pet's behavior and controlling the pet when subjected to stress. Further, the wearable device is operable to automatically activate the vibrating element for controlling the pet, when subjected to high-agitation levels and displaying unruly or undisciplined behavior (like, aggressively pulling the dog owner or barking at someone). The vibrating element is activated as soon as the dog starts pulling and stops as soon as the dog stops. The vibrations are deeply unpleasant for the dogs and serve to curtail prolonged pulling and aggressive behavior. Specifically, the dog associates the pulling with an unwelcome sensation and accordingly curtails prolonged pulling. The wearable device can be fitted or coupled to a standalone leash, a collar, a harness, or a vest.

The embodiments illustrated and described herein as well as embodiments not specifically described herein but within the scope of the aspects of the invention constitute an exemplary wearable device and a system for monitoring and controlling behavior of a pet.

The benefits and advantages described above may relate to one embodiment or may relate to several embodiments. The embodiments are not limited to those that solve any or all of the stated problems or those that have any or all of the stated benefits and advantages.

Aspects of any of the examples described above may be combined with aspects of any of the other examples described to form further examples without losing the effect sought.

The above description is given by way of example only and various modifications may be made by those skilled in the art. The above specification, examples and data provide a complete description of the structure and use of exemplary embodiments. Although various embodiments have been described above with a certain degree of particularity, or with reference to one or more individual embodiments, those skilled in the art could make numerous alterations to the disclosed embodiments without departing from the spirit or scope of this specification.

1. A wearable device for monitoring and controlling behavior of a pet, the wearable device comprising:
a wearable element configured to be worn by the pet;
at least one sensor arranged on the wearable element, wherein the at least one sensor comprises at least one biometric sensor operable to sense biometric-parameters based on biometric signals of the pet;
a processor arranged on the wearable element and operatively coupled to the at least one sensor, the processor configured to receive the biometric-parameters from the at least one biometric sensor and communicate the biometric-parameters to a user device for enabling a user to monitor and control behavior of the pet;
wherein the at least one sensor further comprises at least one force sensor arranged on the wearable element, the at least one force sensor operable to sense force signals when the pet applies a pull force on the wearable element coupled to a leash;
further comprising at least one vibrating element arranged on the wearable element and operatively couple to the processor, wherein the processor is configured to receive the force signals from the at least one force sensor to automatically activate the at least one vibrating element for controlling behavior of the pet;
wherein the pull force is an adjustable pressure force associated with the at least one vibrating element for controlling behavior of the pet including applying the adjustable pressure force when the animal performs at least one of undisciplined barking, forcibly pulling an owner; and
wherein the at lease one vibrating element is at least one of a vibrating sensor, a portable vibrating fence, and a vibrating bell.

2. The wearable device according to claim 1, wherein the wearable element comprises one of a vest, a harness, or a collar.

3. (canceled)

4. (canceled)

5. The wearable device according to claim 4, wherein the at least one sensor further comprises at least one sound sensor arranged on the wearable element and operatively couple to the processor, the at least one sound sensor operable to sense a barking sound of the pet.

6. The wearable device according to claim 5, wherein the processor is configured to receive the barking sound from the at least one sound sensor to automatically activate the at least one vibrating element for controlling behavior of the pet.

7. The wearable device according to claim 6, wherein the processor is configured to correlate data of at least two of the at least one biometric sensor, the at least one force sensor and the at least one sound sensor prior to automatically activating the at least one vibrating element.

8. The wearable device according to claim 6, further comprising a speaker arranged on the wearable element and operatively coupled to the processor, wherein the processor is operable to activate the speaker to generate a warning sound prior to activating the at least one vibrating element.

9. The wearable device according to claim 8, further comprising a light source arranged on the wearable element and operatively coupled to the processor, wherein the processor is operable to activate the light source upon activating the at least one vibrating element.

10. A system for monitoring and controlling behavior of a pet, the system comprising:
a wearable device comprising
a wearable element configured to be worn by the pet,
at least one sensor arranged on the wearable element, wherein the at least one sensor comprises at least one biometric sensor operable to sense biometric-parameters based on biometric signals of the pet, and
a processor arranged on the wearable element and operatively coupled to the at least one sensor, the processor is configured to receive the biometric-parameters from the at least one biometric sensor;
a user device communicably coupled to the wearable device for receiving the biometric-parameters, the user device notifies the biometric-parameters to a user for enabling the user to monitor and control behavior of the pet;
wherein the at least one sensor of the wearable device further comprises at least one force sensor arranged on the wearable element and operatively coupled to the processor, the at least one force sensor is operable to sense force signals when the pet applies a pull force on the wearable element coupled to a leash;
wherein the wearable device further comprises at least one vibrating element arranged on the wearable element and operatively coupled to the processor, the processor is configured to receive the force signals from the at least one force sensor to automatically activate the at least one vibrating element for controlling behavior of the pet; and
wherein the at lease one vibrating element is at least one of a vibrating sensor, a portable vibrating fence, and a vibrating bell.

11. The system according to claim 10, wherein the wearable device further comprising a communication interface for enabling communication coupling between the wearable device and the user device.

12. (canceled)

13. (canceled)

14. The system according to claim 13, wherein the at least one sensor of wearable device further comprises at least one sound sensor arranged on the wearable element and operatively coupled to the processor, the at least one sound sensor is operable to sense a barking sound of the pet.

15. The system according to claim 14, wherein the processor of the wearable device is configured to receive the barking sound from the at least one sound sensor to automatically activate the at least one vibrating element for controlling behavior of the pet.

16. The system according to claim 14, wherein the processor of the wearable device is configured to correlate data of at least two of the at least one biometric sensor, the at least one force sensor and the at least one sound sensor prior to automatically activating the at least one vibrating element.

17. The system according to claim 16, wherein the wearable device further comprises a speaker arranged on the wearable element and operatively coupled to the processor, the processor is operable to activate the speaker to generate a warning sound prior to activating the at least one vibrating element.

18. The system according to claim 17, wherein the wearable device further comprises a light source arranged on the wearable element and operatively coupled to the processor, the processor is operable to activate the light source upon activating the at least one vibrating element.

19. The system according to claim 17, further comprising a prohibitory sensor node and a portable vibrating fence communicably coupled to the wearable device or the user device, wherein the prohibitory sensor node and the portable vibrating fence are operable to prohibit the pet against accessing a prohibited part of the house.

20. The system according to claim 19, further comprising a bell arrangement communicably coupled to the user device, wherein the bell arrangement is operable to generate a tone when the pet sits in front of the bell arrangement for a pre-determined time period and notify the user device that the pet is sitting in front of the bell arrangement.
